(19) United States
(12) Patent Application Publication
(10) Pub. No.: US 2019/0275096 A1
(43) Pub. Date: Sep. 12, 2019

(54) Food-based lutein and zeaxanthin from Momordica Cochinchinensis Spreng, method and composition

(71) Applicant: Le Thuy Vuong, Elk Grove, CA (US)

(72) Inventor: Le Thuy Vuong, Elk Grove, CA (US)

(21) Appl. No.: 15/916,252

(22) Filed: Mar. 8, 2018

(51) Int. Cl. A61K 36/42; A61K 31/047; A61K 31/015; A23L 33/105

(52) U.S. Cl. CPC A61K 36/42; A61K 31/047; A61K 31/015; A23L 33/105; A23V 2002/00; A61K 2236/37; A61K 2236/13; A61K 2236/17; A61K 2236/15

(57) ABSTRACT

Lutein and Zeaxanthin (Lut&Zea) are essential for eye health and have been used to prevent or slow down progress of age-related macular degeration (AMD), dry macular degeneration, night blindness or other vision impair. This present invention describes novel applications of a formulation rich in lutein, zeaxanthin, beta-carotene and antioxidants for eye care. The formulations and associated method are characterized by extract from Momordica cochinchinensis Spreng, and a lipid profile high in plant retinol and vitamin E, synergistically improve bioavailability and stablize lutein and zeaxanthin. The novelty of this invention is in the mixture of antioxidants with macular carotenoids (lutein and zeaxanthin) and pre-vitamin A. This invention does not involve the use of toxic solvents, provides a food-based solution to the populations at risk without long-term negative effects to subjects, nor causing more damage to the environment.

### PARENT CASE TEXT AND RELATED APPLICATION

This application is a continuation-in-part of U.S. Pat. No. 6,770,585 B2 issued Aug.3, 2004.

### FIELD OF INVENTION

This invention relates to methods and formulation to extract lutein and xeazanthin from a fruit. Accordingly, the invention involves the fields of botany, nutritional, eye care, nutraceuticals, nutrition, sport nutrition, natural ingredients, health sciences, medicine, and animal feed.

### BACKGROUND AND PRIOR ART

Momordica cochinchinensis (Lour.) Spreng, belongs to the melon family (Cucurbitaceae) and is indigenous to Southeast Asia and consumed there for dietary as well as medicinal uses. However, it is almost unknown to Western cultures. In Vietnam, this plant is called “Gac”, and the seed membrane (seed pulp or aril), and seeds of the ripe fruit is widely used, while the rind and peels are discarded. The rind (mesocarp, meat) of the green fruit is used in some regions in South East Asia in vegetable dishes. As the fruit ripens, the mesocarp becomes thicker, spongy and yellow-orange. The cavity is divided into cartilaginous chambers containing bright red fleshy seed pods. Mass distribution of a ripe Gac fruit is as follows: 18% of skin, 49% of pulp, 14% of seeds and 18% of aril (seed pulp). Concentrations of vision carotenoids in each part of Gac fruit are varied depending on stages of ripeness, varieties of Gac fruit and analytical method. In FIG. 1, the concentrations of bioactive compounds are presented. In summary, the skin of the fruit contains highest amount of lutein and the aril highest β-carotene and small amount of zeaxanthin. The pulp contains lutein and β-carotene. Aril contains the most antioxidants particularly fat soluble such as vitamin E, lycopene, also polyphenolic compounds and flavonoids.

Gac fruit has many potential health benefits for its high carotenoid content. Carotenoids are natural pigments that occur in bacteria, plants, fungi, and animals. Carotenoids comprise a class of hydrocarbons (carotenes) and their oxygenated derivatives (xanthophylls). Most carotenoids are 40 carbon isoprenoid compounds called tetraterpenes. Carotenoids having fewer than 40 carbons can result from loss of carbons within the chain (norcarotenoids) or loss of carbons from the end of the molecule (apocarotenoids). Longer carotenoids, homocarotenoids (C45-050), are found in some bacterial species. The alternating double bonds along the backbone of carotenoid molecules form a polyene chain, which imparts unique qualities to this group of compounds. This alternation of single and double bonds also allows a number of geometrical isomers to exist for each carotenoid. Carotenoids exist in both trans- and cis-isomeric forms of which the former can be converted to the latter by exposure to light, heat, or chemical reaction. Carotenoids cannot be synthesized by humans, hence, must be obtained from dietary sources. For humans the most important sources for carotenoids are plants. These are primarily highly pigmented red, orange, and yellow fruits and vegetables. Exemplary carotenoids found in fruits and vegetables are lycopene, .beta.-carotene, lutein, zeaxanthin, lutein plus zeaxanthin, .beta.-cryptoxanthin, and .alpha.-carotene. Carotenoids are important factors in human health. The essential role of .beta.-carotene as an important dietary source of vitamin A has been known for many years. More recently, protective effects of carotenoids against serious disorders such as cancer, heart disease, and degenerative eye disease have been recognized. Lycopene is typically the carotenoid consumed in greatest amounts in Western diets. Per capita intakes in Europe and North America average from 1.6 to more than 18 mg lycopene per day.

Processed foods are frequently fortified with carotenoids such as lycopene to increase nutritive value and/or enhance attractiveness. Carotenoids act as antioxidants and/or singlet oxygen quenchers or as free radical scavengers. With 11 conjugated double bonds and no cyclic groups, lycopene is the most potent biological antioxidant. Of the many health benefits attributed to the consumption of carotenoids, these provide the greatest clinical depth. Lycopene is an important antioxidant and free radical scavenger. Free radicals can cause damage both to the structure and to the function of cell membranes, DNA, and proteins. This damage has been linked to the onset of many degenerative diseases such as cancer, atherosclerosis, cataracts, and age-related macular degeneration as well as to premature aging. The free radical quenching constant of lycopene has been demonstrated to be more than twice that of .beta.-carotene. Lycopene is incorporated into lipoproteins. There it acts to decrease the oxidation of cholesterol, helping to prevent vascular damage. Lycopene in the blood has been shown to be inversely proportional to the incidence of prostate tumors. Research shows lycopene may provide protection against a broad range of epithelial cancers. Carotenoids are broadly used as dietary supplements due to their antioxidant potential, and lycopene has a strong presence in the nutraceuticals market.

A method using no chemicals to extract beta-carotene from Momordica cochinchinensis (Gac fruit) was described by Vuong in U.S. Pat. No. 6,770,585 filed Aug. 2, 2002. This patent claimed a process to produce an oil rich in carotenoids to be used as a safe source of dietary supplement of vitamin A. A process filed subsequently by Ishida et al. (U.S. Pat. No. 7,572,468) described a process using chemical solvent to extract carotenoids from plants, including Gac fruit. In US Patent 20070212433) Smidt et al. described beverage compositions using Gac fruit in combination with Cili fruit extract, Siberian pineapple extract, and a Wolfberry extract.

In U.S. Pat. No. 5,942233 Chang, Shan described the use of Momordica cochinchinensis as one of the component in a paste that is useful for re-establishing of vital energy invigorating of blood circulation. U.S. Pat. No. 8,017,147, described compositions for the prevention of cardiovascular disease, alzheimer's disease, diabetes, and regulation and reduction of blood sugar and insulin resistance. The inventor mistakenly listed “Momordica chinensis” as a synonym of Bitter Melon (Momordica charantia). U.S. Pat. No. 8,668,942 (Vuong) described the use of the oil extract from Momordica cochinchinensis in skin anti-oxidant enhancing formulations. Foreign invention filed on the use of Momordica cochinchinensis to treat skin disorders are either based on components from inside the seeds or from the root of the plant, or a mixture of many different plants.

All of the patents listed above described only the use of the aril. In this patent, all parts of the fruit are used to maximize the concentrations of vision carotenoids. Lutein is more concentrated in the rind and pulp, while the aril contains zeaxanthin β-carotene and antioxidants.

Macular pigment (MP) is composed of the two dietary carotenoids lutein (L) and zeaxanthin (Z), and is believed to protect against age-related maculopathy (ARM). Lutein and zeaxanthin, the two carotenoids found in vegetables and fruits are important for eye health (Abdel-Aal el-S M, Akhtar H, Zaheer K, Ali R.), particularly in the prevention of age-related macular degeneration (AMD). Age-related macular degeneration (AMD) is an ophthalmologic disease which usually affects older adults and represents the leading cause of legal blindness in Europe and the United States of America. Lutein (L) and zeaxanthin (Z) are concentrated at the macula, where they are collectively known as macular pigment (MP), Lutein (L) and zeaxanthin (Z) are also believed to play a major role in protecting retinal tissues against oxidative stress. Whilst the exact pathogenesis of age-related maculopathy (ARM) remains unknown, the disruption of cellular processes by oxidative stress may play an important role. Manipulation of dietary intake of L and Z has been shown to augment MP, thereby raising hopes that dietary supplementation with these carotenoids might prevent, delay, or modify the course of ARM. This article discusses the scientific rationale supporting the hypothesis that L and Z are protective against ARM, and presents the recent evidence germane to this theory. There is also epidemiological evidence that increasing lutein and zeaxanthin intake lowers the risk of cataract development.

Consumption of more than 2.4 mg of xanthophylls daily from foods and supplements was significantly correlated with reduced incidence of nuclear lens opacities, as revealed from data collected during a 13- to 15-year period in the Nutrition and Vision Project (NVP) (San Giovanni J P et al.).

The macular pigments are predominantly composed of three carotenoids: lutein, zeaxanthin, and meso-zeaxanthin. These carotenoids are concentrated and distributed in a selective manner. The properties of these pigments are further explored along with their methods of uptake, stabilization, and storage. The macular xanthophille (lutein and zeaxanthin), as well as all carotenoids, are sensitive to light, oxygen and heat. They are lipophylic (soluble in fat), therefore dietary rich in fatty acids have been shown to improve bioavailability of lutein (Nidhi B, Mamatha B S, Baskaran V, Eur J Nutr. 2013 Mar. 30).

The dual nature of these pigments as filters and macular xanthophylls are elaborated upon in relation to their protective effects upon the macula, specifically in age-related macular degeneration. Evidence suggests that increased levels of macular pigment are correlated with a decreased risk of age-related macular degeneration. Many have sought to exploit this therapeutic relation. Studies reveal that oral supplementation with lutein and zeaxanthin can increase the levels of macular pigments in the retina and plasma. The effects of such supplementation on actual ocular function have yet to be fully addressed. New and standardized methods of assessing macular pigment density are discussed and future areas of research to further our understanding of macular xanthophylls as they pertain to age-related macular degeneration are highlighted.

There is ample epidemiological evidence that the amount of macular pigment is inversely associated with the incidence of AMD, an irreversible process that is the major cause of blindness in the elderly. The macular pigment can be increased in studies by either increasing the intake of foods that are rich in lutein and zeaxanthin, such as dark-green leafy vegetables, or by supplementation with lutein or zeaxanthin (Krinsky N I, Landrum J T, Bone R A).

Due to their important roles in maintaining eye sight, methods to extract lutein and xeazanthin continue to be sought.

Lutein and zeaxanthin could be obtained either from biosynthesis, or extracted from natural sources, such as corn, marigolds, kale, spinach. Methods Involving the use of solvents such as textrahydrofuran (THF), methanol, hexan, ethanol are described by Khachik et al., King et al.) have been published.

In U.S. Pat. No. 6,169,217 B1, Munir Cheryan described a method to extract xanthophylls from corn using raw corn or a corn by-product as a source. This method used alcohol to produce an alcohol-corn slurry, following with centrifugation and filtration to produce a clear filtrate from the alcohol-slurry. After that, membrane filtration is used to separate purified xanthophylls.

Methods to extract oil and zein from corn using ethanol were described by Munir Cheryan (U.S. Pat. No. 6,433,146 B1). Corn solid are separated from the ethanol (60-100%), oil and zein mixture are produced. Ethanol, oil and zien mixture are membrane filtered to get zein in the mixture, and passed an oil and ethanol mixture. Zien or oil is then selected to be separated for corn product.

Formulations containing certain quantities of zeaxanthin stereoisomer bio-synthesized by Flavobacterium multivorum which has been genetically modified was described by Garnette et al. in U.S. Pat. No. 5,747,544 A.

Methods of extracting lutein from corn or marigolds have been described in many US patents, some of which are listed below: U.S. Pat. No. 3,069,443 (Sep. 17, 1959 and Dec. 18, 1962), U.S. Pat. No. 3,523,138 (Mar. 1, 1965 and Aug. 4, 1970 by Eastman Kodak Co.). U.S. Pat. No. 5,382,714 (Mar. 17, 1994 and Jan. 17, 1995) by The Catholic University Of America described a process for isolation, purification, and recrystallization of lutein from saponified marigold oleoresin. U.S. Pat. No. 5,602,286 filed on Jun. 7, 1995 and Feb. 11, 1997 by Cargill, Incorporated described a process for recovering xanthophylls from corn gluten. U.S. Pat. No. 4,851,339 (Apr. 1, 1986 and Jul. 25, 1989) by Hills; Christopher B described a method to extraction pigments from algae and vegetables. U.S. Pat. No. 5,648,564 filed on Dec. 21, 1995 and Jul. 15, 1997 by Kernin Industries, Inc. described a process for the formation, isolation and purification of comestible xanthophyll crystals from plants. U.S. Pat. No. 5,747,544 filed on Oct. 31, 1995 and May 5, 1998 by Applied Food Biotechnology, Inc. described a method of using pure 3R-3′R stereoisomer of zeaxanthin to treat or prevent retinal degeneration in humans. U.S. Pat. No. 5,847,238 filed on Nov. 29, 1996 and Dec. 8, 1998) by Cargill, Incorporated described a processes for recovering xanthophylls from corn gluten meal. U.S. Pat. No. 5,876,782 filed on May 14, 1997 and Mar. 2, 1999 by Kemin Industries, Inc, described a method for the conversion of xanthophylls in plant material. U.S. Pat. No. 5,998,678 (Feb. 25, 1997) by Investigaciones Quimicas Y Farmaceutics, S.A. described a process for preparing carotenoid pigments. U.S. Pat. No. 6,169,217 B1 filed on October 1999 by Munir Cheryan described a method for extracting xanthophylls from corn. U.S. Pat. No. 7,671,242 filed on Dec. 21, 2005 and Mar. 2, 2010 by Board Of Supervisors Of Louisiana State University And Agricultural And Mechanical College described a method to Isolate lutein from aflatoxin-contaminated plants and plant products. U.S. Pat. No. 7,943,804 on Feb. 20, 2007and May 17, 2011 from Board Of Supervisors Of Louisiana State University And Agricultural And Mechanical College described method to extract lutein from ozone-treated plant sources. U.S. Pat. No. 8,236,929 filed on May 8, 2007 and Aug. 7, 2012 the Board Of Trustees Of The University Of Illinois described method and system for production of zein and/or xanthophylls using chromatography.

All methods listed above involved the use of alcohol (ethanol, butanol, methanol, propanol), and/or tetrahydrofuran (THF) either in the extraction, purification, separation or saponification. Other methods using genetically modified plant breeding, or bacterial fermentation may not be applicable at industrial scale. None of those methods use Momordica cochinchinensis Spreng. as a source for lutein and zeaxanthin. Further, the products do not contain antioxidants which are important in the protection of the retina from oxidative damage. As for methods that use chemical solvents, although there is step to remove the solvents, there is no certainty that no residues of those chemicals in the extracted products. Long term and repeat consumption of those products, albeit small amount, might lead to irreversible negative health outcomes. In humans, L and Z are found in the retina, and adipose tissues. In chicken (fed with feed supplemented with lutein and zeaxanthin), those compounds are accumulated in beaks, skin, fat cells and eggs. Although the chemicals used during the extraction process might be excreted completely or retained in a non detectable amount, consumption of large amount of food containing chemical solvents in long term, exaggerated by other processed foods might lead to detrimental health outcomes. In fact, some of the solvents used in those methods are considered carcinogen by OSHA and EPA. Chronic toxic effects associated with accumulation of acetaldehyde (a metabolite of ethanol) in the liver has been reported (National Research Council (NRC, 1983) EPA Guidelines and Risk Assessment Forum (U.S. EPA, 1986), Fetal Alcohol Spectrum Disorder (FASD), Braun, 1996) is one known problem of chronic gasoline product exposure (ethanol, benzene, toluene, methanol). Neurological damage, mutagenicity, organ failure due to long term exposure to THF, Ethanol, Hexane, methanol, have been reported (see guidelines from NTP, IARC, OSHA and ACGIH). One of the common solvents used in the above methods is ethanol, a strong alcohol. Ethanol is inflammable and combustion causes hazardous decomposition products. It can be absorbed through skin and cause irritation. Eye contact causes irritation. Inhaling into lungs is hazard, resulting in chemical pneumonia, severe lung damage, respiratory failure and even death. Ingestion of ethanol can cause gastrointestinal disturbances, including irritation, nausea, vomiting and diarrhea and central nervous system damage similar to alcohol intoxication. Occupational health risk from inhalation of those chemicals is another concern (See Chapter 4—Hazard Identification, EPA Toxicological Review of THF, 2/2012; OSHA Safety and Health Guideline for Hexane). In laboratory setting, the evaporation of those solvents can be carried out under fume hood, with extreme cautions, however, in commercial setting, disposing of large quantities of those toxic chemicals into the air or water source is not economical, nor sustainable.

### OBJECTS AND ADVANTAGES

The novelty of this invention is in the use of a fruit to produce a composition rich in compounds that have been known to prevent and slow the progress of macular degeneration and other eye diseases. Furthermore, due to the fruit matrix rich in oil, the active ingredients are bioavailable and stable. In addition to the active ingredients, the composition contains antioxidants which provide additional protection to eye health. The use of water and CO2 extraction method result in a clean and safe product. In summary, advantages of this invention are in the source of the xanthophylls, delivery method to increase bioavailability, and extraction method involving no organic solvents. The methods require no cleaning step (removal and disposal of solvents), which might incur loss of the xanthophylls, risk to consumers due to incomplete removal of the solvents, and risk to workers due to exposure to the toxic solvents. Further the use of toxic solvents in larger scale will have negative impact to the environment.

### SUMMARY OF INVENTION

This invention describes a method to composition containing lutein, zeaxanthin and beta-carotene (vision carotenoids) that can be used to prevent and slow down the progress of age-related macular degeneration, dry macular degeneration, night blindness and other eye diseases. The invention describes the method to extract the vision carotenoids from the fruit Momordica cochichinensis Spreng., also known as gac (Vietnamese) or spike gourd, or redmelon. The fruit has been used for generations in Asia as a vegetable. The peel, rind, pulp and seed pulp of the fruit are edible and contain high concentrations of carotenoids, particularly lycopene and vision carotenoids. In addition, pulp and aril also contains oil, vitamin E and other antioxidants. This invention provides a safe, bio-available and stable source of vision carotenoids for eye health.

### BRIEF DESCRIPTION OF INVENTION

The present invention describes novel use of the fruit Momordica cochinchinensis as a food source of lutein and zeaxanthin, and in the method of extraction without any organic solvents. Lutein and zeaxanthin, namely the macular xanthophylls, are pigments found in the retina, and have been shown to be linked to the prevention and slowing the progress of age related macular degeneration. They can he obtained solely from diet. Momordica cochinchinensis (mc) is a variety of melon, indigenous to Asia, Australia, central and Latin America and contains high concentrations of lutein and zeaxanthin, in addition to antioxidants. This invention describes method to extract the macular xanthophylls from raw Momordica cochinchinensis, and food-based application that resulted in increase of plasma level of zeaxanthin of subjects of a large clinical trial. This invention provides a scalable method to obtain lutein and zeaxanthin that would be safe to consumers, also will not cause damage to workers, or to the environment. The products from this invention contain not only lutein and zeaxanthin but also beta-carotene and antioxidants.

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting.

Definition of Terms

In describing and claiming the present invention, the following terminology will be used. The singular forms “a,” “an,” and “the” include plural referents unless the context clearly dictates otherwise. Thus, for example, reference to “a carrier” includes reference to one or more of such carriers, and reference to “an excipient” includes reference to one or more of such excipients.

As used herein, “formulation” and “composition” may be used interchangeably herein, and refer to a combination of two or more elements, or substances. In some embodiments a composition may include an active agent and a carrier.

As used herein, “effective amount” refers to an amount of an ingredient, namely composition, is sufficient to be able to detect by available analytical tool by qualified persons.

The term Redmelon or Gacmelon or Gac or “Gac fruit” or “Gac plant” may be used interchangeably herein.

The term “Gac Fruit,” “Gac Extract,” “Gac fruit extract,” “Gac Fruit Puree,” “Gac powder,” “Gac oil,” “Gac fruit oil” refer to a product made from the fruit of all strains and hybrids of the plant Momordica cochinchinensis Spreng, or of plants significantly related thereto, grown anywhere in the world including blends, mixtures, and combinations of such strains and relatives.

As used herein, the term “conventional carotene drink” or “conventional beverage” refers to beverage in any form which do not contain Gac fruit or a Gac fruit extract.

### BRIEF DESCRIPTIONS OF DRAWINGS

FIG. 1. lists names and concentrations of bioactive compounds in each part of the fruit Momordica cochinchinensis Spreng.

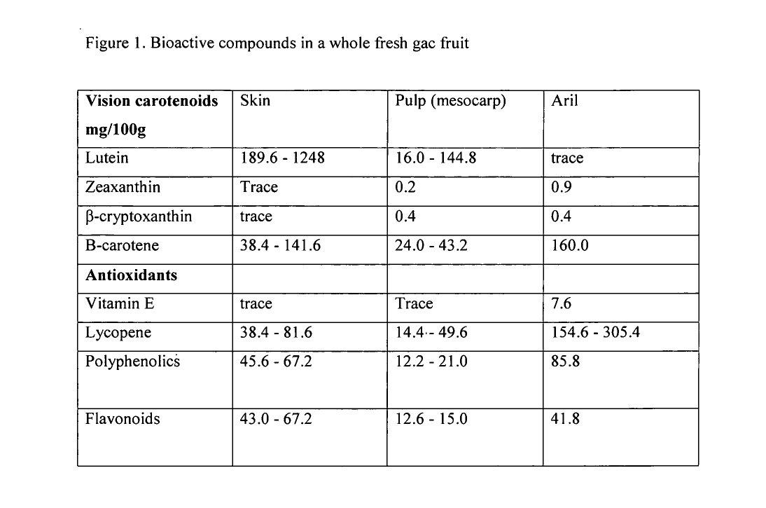

### DETAILED DESCRIPTION OF THE INVENTION

The present invention describe method and formulation containing high concentrations of lutein and zeaxanthin for the prevention of AMD, dry macular degeneration, or any visual impair by long-term exposure to blue light (computer screen) or age-related. All parts of a pipe Gac fruit contains lutein, zeaxanthin and beta-carotene and antioxidants except in the seeds, which should be removed. The soft rind, pulp and aril can be used to produce a mixture rich in visual carotenoids.

In one embodiment, the mixture is dried in a box dryer until water content is about 20%, then loaded into an extractor using CO2 as solvent. The extracts are further mixed with oil extract from the aril to make an oil solution containing 48% of lutein and 10% of zeaxanthin. The solution can be encapsulated to protect carotenoids, or used directly as a natural supplement for eye health.

In another embodiment, fresh Gac mixture can be mechanically blended into puree and can used directly in foods or beverage or can further be subjected to a reduction in particle size by go through a sieve.

The Gac slurry can also be converted into a solid particulate form, having a moisture content of less than 10 wt %. The dried powder is subjected to a second reduction in particle size, by a high speed centrifuge where the residual solids are discharged and the liquid directed to an evaporator.

In one embodiment, the puree is strained to produce a fruit mixture with orange color. The mixture can be sweetened by sugar, honey, or fruit extract or other natural sweeteners suitable for at least certain exemplary embodiments include, for example, sucrose, liquid sucrose, fructose. Sweetened gac puree can be made into a syrup to add to water and used in daily drink, or as a function drink to improve vision.

In another embodiment, the fruit mixture is added to purified water to make a sport drink containing vision carotenoids, and antioxidants. In another embodiment, the fruit mixture with or without sweetener is added to coconut drink for added antioxidants and vision carotenoids. In another embodiment, lemon juice, or ascorbic acid is added to improve taste. In another embodiment, CO2 is added to the water+fruit mixture to produce a carbonated drink for eye health.

### EXAMPLE

A functional drink to improve vision

| Ingredients | Weight (g) | |
|---|---|---|
| Gac puree | 100 | g |
| Liquid sucrose | 485.5 | |
| Citric acid, anhydrous | 3.745 | |
| Concentrated apple extract | 30.0 | |

A sport drink containing visual carotenodis can be prepared by dissolving the above ingredients with vigorous stirring in sufficient water to obtain 1 liter of syrup. The syrup was diluted with carbonated water in a 1 plus 5 “throw” (one volume of syrup plus five volumes of carbonated water) to produce a finished beverage. The beverage was then subjected to tunnel pasteurization at 155.degree. F. for 10 minutes. The beverage made according to this example has a orange color, a very refreshing taste, and highly nutritious. This functional drink can be used every day to improve vision performance. It will be within the ability of those skilled in the art, given the benefit of this disclosure, to select suitable additional or alternative mixture for use in various embodiments of the food and beverage products disclosed here.

Gac mixture either in puree, powder or syrup can be used to add vision carotenoids to yogurt, cakemix or cooked rice. In one embodiment, Gac pulp puree is mixed well with water and rice to produce a red rice dish high in lutein and zeaxanthin.

In another aspect of the present invention, the Gac powder is used in pet food to prevent blindness.

In another aspect of the present invention, the high lutein and zeaxanthin Gac powder is added to chicken feed to produce meat and eggs containing high amounts of vision carotenoids.

It is to be understood that the above-described embodiments are only illustrative of the application of the principles of the present invention. Numerous modifications and alternative formulations can be devised by those skilled in the art without departing from the spirit and scope of the present invention. Thus, while the present invention has been fully described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention. The appended claims are intended to cover all such modifications and alternative embodiments. It should be understood that the use of a singular indefinite or definite article (e.g., “a,” “an,” “the,” etc.) In this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc.

It will be readily apparent to those skilled in the art that various changes and modifications of an obvious nature may be made without departing from the spirit of the invention, and all such changes and modification s are considered to fall within the scope of the invention as defined by the appended claims. Such changes and modifications would include, but not be limited to, the incipient ingredients added to affect the capsule, tablet, lotion, food or bar manufacturing process as well as vitamins, herbs, flavorings and carriers. Other such changes or modifications would include the use of other fruits, bergs or botanical products containing the combinations of the preferred embodiments disclosed above. Many additional modification s and variations of the embodiments described herein may be made without departing from the scope, as is apparent to those skilled in the art. The specific embodiments described herein are offered by way of example only. The scope of the invention is, therefore, indicated by the appended claims rather than by the forgoing description.

### REFERENCES CITED [REFERENCED BY]

U.S. Patent Documents

| | | |
|---|---|---|
| 6,770,585 | Aug. 3, 2004 | Vuong |
| 8,868,942 | Mar. 11, 2014 | Vuong |
| 3,172,770 | March 1965 | Miller |
| 5,245,095 | September 1993 | Graves et al. |
| 5,274,073 | December 1993 | Gruber et al. |
| 5,462,695 | October 1995 | Tsai et al. |
| 5,686,489 | November 1997 | Yu et al. |
| 5,714,658 | February 1998 | Heidlas et al. |
| 5,773,075 | June 1998 | Todd |
| 5,789,647 | August 1998 | Heidlas et al. |
| 5,830,738 | November 1998 | Thomas et al. |
| 5,837,311 | November 1998 | Zelkha et al. |
| 5,871,574 | February 1999 | Kawaragi et al. |
| 5,897,866 | April 1999 | Bombardelli et al. |
| 6,818,239 | November 2004 | Kagan et al. |
| 2003/0052424 | March 2003 | Turner et al. |
| 2003/0061761 | April 2003 | Nanninga et al. |
| 2003/0180435 | September 2003 | Shi |
| 2004/0115260 | June 2004 | Schmid et al. |
| 7,572,468 | Aug. 11, 2009 | Ishida, et al. |
| 6,169,217 B1, | Munir Cheryan | |
| 6,433,146 B1 | Munir Cheryan | |
| 5,747,544 A. | Garnette et al. | |

| | | | |
|---|---|---|---|
| 3,069,443 | Central Soya Company, Inc. | Sep. 17, 1959 | |
| 3,523,138 | Eastman Kodak Co. | Mar. 1, 1965 | Aug. 4, 1970 |
| 4,851,339 | Hills; Christopher B | Apr. 1, 1986 | Jul. 25, 1989 |
| 5,382,714 | The Catholic University Of America | Mar. 17, 1994 | Jan. 17, 1995 |
| 5,602,286 | Cargill, Incorporated | Jun. 7, 1995 | Feb. 11, 1997 |
| 5,648,564 | Kemin Industries, Inc. | Dec. 21, 1995 | Jul 15, 1997 |
| 5,747,544 | Applied Food Biotechnology, Inc. | Oct. 31, 1995 | May 5, 1998 |
| 5,847,238 | Cargill, Incorporated | Nov. 29, 1996 | Dec. 8, 1998 |
| 5,876,782 | Kemin Industries, Inc | May 14, 1997 | Mar. 2, 1999 |
| 5,998,678 | Investigaciones Quimicas Y | Feb. 25, 1997 | Dec. 7, 1999 |
| | Farmaceutics, S.A. | | |
| 6,169,217 B1 | Munir Cherya | October, 1999 | |
| 7,671,242 | Board Of Supervisors Of Louisiana | Dec. 21, 2005 | Mar. 2, 2010 |
| | State University And Agricultural | | |
| | And Mechanical College | | |
| U.S. Pat. No. 7,943,804 | Board Of Supervisors Of Louisiana | Feb. 20, 2007 | May 17, 2011 |
| | State University And Agricultural | | |
| | And Mechanical College | | |
| U.S. Pat. No. 8,236,929 | Board Of Trustees Of | May 8, 2007 | Aug. 7, 2012 |
| | The University Of Illinois | | |

Foreign Patent Documents

| | | |
|---|---|---|
| 53118597 | October, 1978 | JP |

### OTHER REFERENCES

SanGiovanni J P, Chew E Y, Clemons T E, et al. (September 2007). “The relationship of dietary carotenoid and vitamin A, E, and C intake with age-related macular degeneration in a case-control study: AREDS Report No. 22”. Arch. Ophthalmol. 125 (9): 1225-32. doi:10.1001/archopht.125.9.1225. PMID 17846363.
Barker Fm, 2nd (2010). “Dietary supplementation: effects on visual performance and occurrence of AMD and cataracts.”. Current medical research and opinion 26 (8): 2011-23. doi:10.1185/03007995.2010.494549. PMID 20590393.
Stringham, James M., et al. (January/February 2010). “The Influence of Dietary Lutein and Zeaxanthin on Visual Performance”. Journal of Food Science 75 (1): R24-R29. doi:10.1111/j.1750-3841.2009.01447.x. PMID 20492192. Retrieved Jan. 15, 2013.
Abdel-Aal el-S M, Akhtar H, Zaheer K, Ali R. Nutrients. 2013 Apr. 9; 5(4):1169-85. doi: 10.3390/nu5041169. Dietary sources of lutein and zeaxanthin carotenoids and their role in eye health. Guelph Food Research Centre. Agriculture and Agri-Food Canada, Guelph, ON N1G 5C9, Canada. elsayed.abdelaal@agr.gc.ca.
Krinsky N I, Landrum J T, Bone R A. Annu Rev Nutr. 2003; 23:171-201. Epub 2003 Feb. 27. Biologic mechanisms of the protective role of lutein and zeaxanthin in the eye.
Ahmed S S, Lott M N, Marcus D M. Surv Ophthalmol. 2005 March-April; 50(2):183-93. The macular xanthophylls. Department of Ophthalmology, Medical College of Georgia, Augusta, Ga. 30912-3400, USA.
Broekmans W M, Berendschot T T, Klöpping-Ketelaars I A, de Vries A J, Goldbohm R A, Tijburg L B, Kardinaal A F, van Poppel G. Am J Clin Nutr. 2002 September; 76(3):595-603. Macular pigment density in relation to serum and adipose tissue concentrations of lutein and serum concentrations of zeaxanthin. TNO Nutrition and Food Research, Zeist, Netherlands.
Nolan J M, Stack J, O'Donovan O, Loane E, Beatty S. Exp Eye Res. 2007 January; 84(1):61-74. Epub 2006 Nov. 1. Risk factors for age-related maculopathy are associated with a relative lack of macular pigment
O'Connell E, Neelam K, Nolan J, Au Eong K G, Beatty S. Ann Acad Med Singapore. 2006 November; 35(11):821-30. Macular carotenoids and age-related maculopathy. EPMA J. 2010 June; 1(2):245-51.
Hasler P W, Flammer J. Dietary rich in oleic acid improves bioavailability of lutein Eur J Nutr. 2013 Mar. 30.
Vuong, A method of preserving and testing the acceptability of gac fruit oil, a good source of beta-carotene and essential fatty acid, 2003, Food Nutr Bull, 24, 372-3.
Vuong and King, “A method of preserving and testing the acceptability of gac fruit oil, a good source of beta-carotene and essential fatty acids,” 2003, Food and Nutrition Bulletin, 24: 224-230.
Le T. Vuong, Stephen R. Dueker, and Suzanne P Murphy, Plasma .beta.-carotene and retinol concentrations of children increase after a 30-d supplementation with the fruit Momordica cochinchinensis (gac). American Society for Clinical Nutrition. 2002. 872-9.
Hiromitsu Aoki, Nguyen Thi Minh Kieu, Noriko Kuze, Kazue Tomisaka and Nguyen Van Chuyen, Carotenoid Pigments in GAC Fruit (Mamordica cochinchinensis Spreng), Biosci. Biotechnol. Biochem., 2002, 2479-2482, 66(11).
Aaron M Secrest, Kenneth D Sorensen, Carl W Hardie, Katherine M Secrest, Le T Vuong, Byron K Murrayl, Kim L O'Neill Comparative study of total antioxidant activity between the fruit Momordica cochinchinensis (gac) and its major carotenoid constituents; AAAS Conference Poster Presentation, May 2003.
Bailey L H. The Garden of the Gourds. New York: The Macmillian Company, 1937:121-22.
Bendich A, Olson J A. Biological actions of carotenoids FASEB J. 1989 June; 3(8):1927-32.
Schwedhelm E, Maas R, Troost R, Böger R H. Clinical pharmacokinetics of macular xanthophylls and their impact on systemic oxidative stress. Clin Pharmacokinet. 2003; 42(5):437-59.
Shadeque A, Baruah G. Sweet gourd: a popular vegetable of Assam. Indian Farming. 1984; 34:25-35.
Slattery M L, Jacobs D R Jr, Dyer A, Benson J, Hilner J E, Caan B J. Dietary macular xanthophylls and plasma lipids: the Cardia Study. J Am Coll Nut 1995; 14:635-42.
Stahl W, Sies H. Uptake of lycopene and its geometrical isomers is greater from heat-processed than from unprocessed tomato juice in humans. J Nutr 122:2161-2166, 1992.
Stahl W, Heinrich U, Aust O, Tronnier H, Sies H. Lycopene-rich products and dietary photoprotection; Photochem Potobio Sci. 2006 February; 5(2):238-42. Epub 2005 Aug. 12.
Stahl W, Heinrich U, Aust O, Tronnier H, Sies H. Lycopene-rich products and dietary photoprotection. FASEB J. 1989 June; 3(8):1927-32.
Stahl W, Sies H. Photoprotection by dietary carotenoids: Concept, mechanisms, evidence and future development. Mol Nutr Food Res, 2011.

What is claimed is:

1. A process to produce a composition rich in lutein, zeaxanthin and beta-carotene from the fruit Momordica cochinchinensis Spreng (Gac, redmelon);

2. The process in claim 1 comprises of cleaning, de-seeding, homogenizing, and/or pulverizing, and/or drying and/or freeze-drying or spray-drying or lyophilizing, and/or centrifuging, and/or stabilizing and/or purifying and/or pasteurizing to produce a red mixture rich in carotenoids;

3. Process according to claim 1, whereas all parts of a whole fruit (rind, pulp, seeds and aril) are separated, and the seeds are discarded.

4. Process according to claim 3, further comprises of the step of preparing rind, pulp and aril by pulverizing, homogenizing, and drying using either box dryer, lyophilizer, spray-dryer;

5. Process according to claim 2, wherein the above Vision carotenoids (beta-carotene, lutein and zeaxathin) may be further extracted using SFE-CO2, or using non-toxic solvents such as nitrogen, water, or edible oil, or alcohol.

6. Process according to claim 2, whereas an oil may be produced from the red mixture using cold press method, or an oil expeller, or supercritical fluid extraction with CO2 (SFE-CO2), with or without other solvents, such as edible oil, water or nitrogen;

7. Process according to claim 2, whereas the composition may contain at least 428 mg/100 g of Vision carotenoids (beta-carotene, lutein, zeaxanthin), of which at least 44% is lutein;

8. Process according to claim 2, whereas the composition may contain at least 245 mg/100 g of antioxidants in the form of tocopherols, polyphenols and flavonoids;

9. Process according to claim 2, whereas the mixture may be encapsulated, or re-suspense in oil to improve bioactivity and stability, and/or to produce a dietary supplement;

10. Process according to claim 2, whereas the composition can be added to drinkable liquid, or food products;

11. The method in claim 2 wherein the composition might include artificial or natural flavor, or extract, or puree, or concentrate of other fruits to improve flavor;

12. The method of claim 2 wherein the final composition might include excipients selected from the group consisting of flavorants, colorants, stabilizers and preservatives.
